(19) United States
(12) Patent Application Publication
(10) Pub. No.: US 2025/0033848 A1
(43) Pub. Date: Jan. 30, 2025

(54) STERILIZABLE DUAL COMPARTMENT POUCH

(71) Applicant: Oliver Healthcare Packaging Company, New Britain, PA (US)

(72) Inventors: Tommy SMITH, Lynn (IE); Georgianna GALLEGOS, Perkasie, PA (US); Patrick TONREY, Levittown, PA (US); Martin VELAS, Perkasie, PA (US)

(21) Appl. No.: 18/774,005

(22) Filed: Jul. 16, 2024

Related U.S. Application Data
(60) Provisional application No. 63/528,697, filed on Jul. 25, 2023.

(51) Int. Cl. B65D 65/38; B32B 5/02; B32B 15/14; B32B 15/20; B65D 75/26; B65D 75/30; B65D 81/20; B65D 81/26

(52) U.S. Cl. CPC B65D 65/38; B32B 5/022; B32B 15/14; B32B 15/20; B65D 75/26; B65D 75/30; B65D 81/2084; B65D 81/266; B32B 2250/05; B32B 2262/0253; B32B 2307/724; B32B 2307/7242; B32B 2439/02; B65D 2565/387; B65D 2565/388

(57) ABSTRACT

A pouch for holding a medical device. A base layer has a base periphery, a base back end and opposing base outer edges. A breathable first web is on the base layer and adapted to permit a sterilization gas to pass therethrough. A non-breathable middle film is adhered to the first web. A breathable second web is attached to the middle film, and a non-breathable top layer is over the base layer, thereby the pouch includes five layers. A primary seal adheres the base layer and the top layer. Then a secondary seal adheres the middle film and the second web to the base layer. The secondary seal is coextensive with the primary seal but then extends a partial width of the primary seal to thereby form a setback, wherein the setback allows the first web, the middle film and the second web to be formed in a single sealing step.

### CROSS REFERENCE TO RELATED APPLICATIONS

The present application claims the benefit of provisional application Ser. No. 63/528,697, filed Jul. 25, 2023, the contents of which are incorporated herein by reference.

### FIELD OF THE INVENTION

The present invention relates to pouch-type packaging. In particular, provided is a pouch well-suited for storing sterilized medical devices.

### DESCRIPTION OF THE RELATED ART

Packaging for medical devices and particular for Class III medical devices must typically be sealed and contained within a sterilized environment while also having a regulated, ambient environment to allow for oxygen to maintain the efficacy of certain drugs that may be contained in or coated on the device. But this ambient condition also risks a path for microbes and moisture. Thus, such packages include breathable webs to allow for the ingress of a sterilization gas and multiple compartments, e.g. one compartment for containing the device, and another compartment for receiving a desiccant. In this manner, exposure of the device to excess oxygen, moisture, and light is minimized, but the package is also sterilized and breathable.

Drawbacks in the prior art exist inasmuch as when breathable layers are utilized, excess oxygen ingress may still occur unless the sealing of the packaging is ongoingly effective. Prior art devices attempt to increase the integrity of the seals by double-sealing the layers at the peripheral edges. For example, an outer seal along a boundary of the package fixes a first layer to a second layer to make one compartment, then an inner seal near the edge fixes a third layer to the first layer to form the second compartment. However, this double-seal requires extra manufacturing steps and increases the probability of ingress. The seals further do not entirely overlap across all of the lengths.

There is a need then for a multi-web pouch which allows the breathable material to be effectively sealed at the outer edges in a single step, thereby encapsulating five webs of material in one continuous seal.

### SUMMARY OF THE INVENTION

It is an objective of the present invention to provide a pouch which provides for device access from only one side, in that a peel-open feature is presented on one side of the compartment to allow for aseptic presentation of the device.

It is further an objective to provide a multi-web pouch which allows the breathable material to be effectively sealed at the outer edges in a single step, thereby encapsulating five webs of material in one continuous seal.

It is further an objective to increase the efficacy and strength of the peripheral seal of the pouch.

Accordingly, comprehended is a dual compartment pouch formed from five separate layers of materials, and which has a peripheral edge seal that includes direct adherence of impermeable outer layers across a first part of its width to help maintain seal strength to resist egress while also capturing one or more webs of a breathable material between those two outer layers across a second part of that same peripheral seal's width, such that the pouch has a single, continuous peripheral seal.

More particularly, comprehended is a pouch, comprising: a base layer having a base periphery, a base back end and opposing base outer edges; a breathable first web on the base layer having a first web back end and a first web front end and adapted to permit a sterilization gas to pass therethrough; a non-breathable middle film adhered to the first web having a first edge, a second edge and opposing film side edges; a breathable second web attached to the middle film having a web rear edge, a web front edge and opposing web side edges; and, a non-breathable top layer over the base layer, thereby the pouch includes five layers. The top layer has a top periphery, a film rear edge, a film front edge and opposing top layer side edges, thereby forming a back edge defined between the first web back end and the base back end, wherein a sterile main pocket is formed between the base layer and the top layer.

A primary seal adheres the base layer and the top layer along the top periphery and the base periphery, the primary seal overlapping an entirety of the base outer edges to seal the top layer side edges. A secondary seal adheres the middle film and the second web to the base layer at the base outer edges. The secondary seal is coextensive with the primary seal at an overlap location, the secondary seal extending a partial width of the primary seal at a partial overlap portion distal to the overlap location to thereby form a setback from the base outer edges, wherein the setback allows the first web, the middle film and the second web to be formed in a single sealing step.

A process of manufacturing forms a pouch. The pouch comprises a base layer; a breathable first web on said base layer having a first web back end and a first web front end and adapted to permit a sterilization gas to pass therethrough; a non-breathable middle film adhered to said first web; a breathable second web attached to said middle film having a web front edge; a non-breathable top layer over said base layer; a primary seal adhering said base layer and said top layer along a top periphery and a base periphery; and, a secondary seal adhering said middle film and said second web to said base layer. The process comprises simultaneously feeding said base layer, said breathable first web, said non-breathable middle film, said breathable second web, and said non-breathable top layer to form a film assembly. The process also comprises forming said primary seal and said secondary seal in a single sealing step by applying pressure and heat to said film assembly, said secondary seal overlapping said primary seal an entire length of said primary seal between said web front edge and said first web front end at said top periphery and said base periphery, thereby forming said pouch.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows a diagrammatic representation in top elevation view of the pouch.

FIG. 2 shows one embodiment of detail A of FIG. 1.

FIG. 2a shows another embodiment of detail A of FIG. 1.

FIG. 3 depicts a cross-sectional view of the pouch of FIG. 1 through a vertical plane.

FIG. 3a depicts a cross-sectional view of another embodiment of the pouch of FIG. 1 through a vertical plane.

FIG. 4 shows an exploded, perspective view of a film assembly without the top layer.

FIG. 5 shows the same perspective view of the film assembly as FIG. 4 but with the pouch partially assembled with its layering.

FIG. 6 shows a top elevation view of the pouch.

FIG. 7 shows perspective views of the front and back of the pouch.

Wherever possible, the same reference numbers will be used throughout the drawings to represent the same parts.

### DETAILED DESCRIPTION OF THE INVENTION

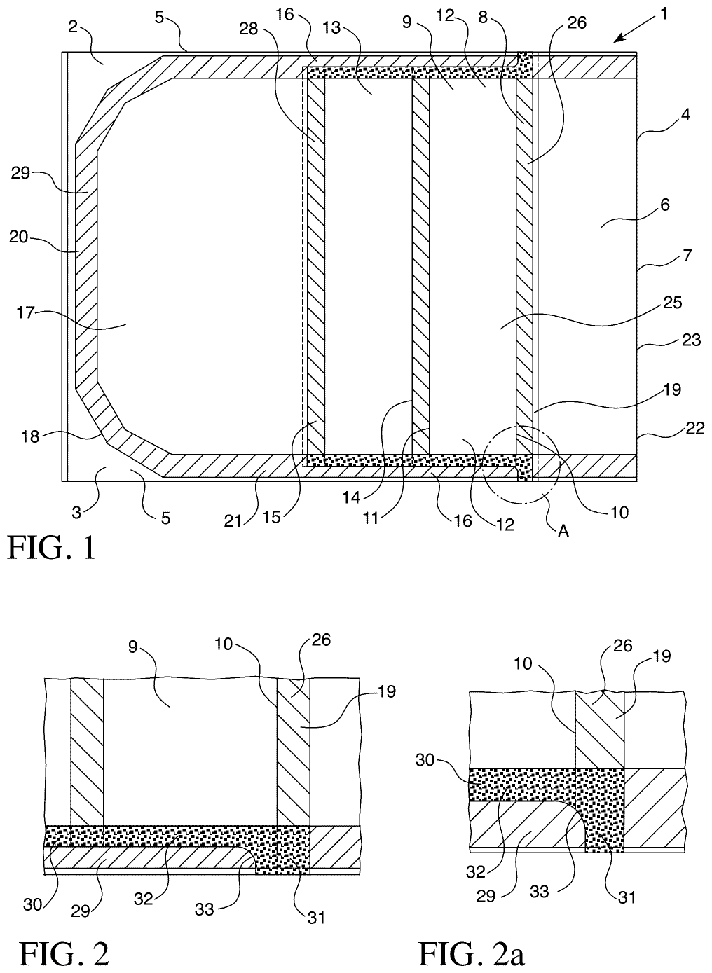

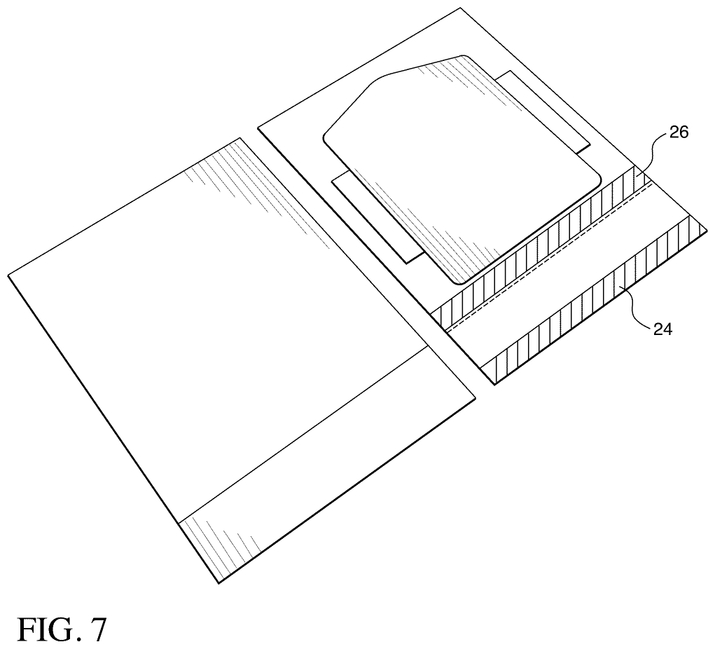

With reference then to FIGS. 1-7, shown is a package, termed herein pouch 1. The pouch 1 is well-suited to contain a medical device within both an ambient, stable environment as well as an environment which is sterile. For example, many medical devices must be sterilized and also maintained in a substantially moisture-free environment, further within a stable environment wherein excess oxygen and potential gas containments do not ingress into the packaging. This could impact the efficacy of any drugs incorporated into or coated onto such devices. Accordingly, there needs to be an ideal combination of an ambient, oxygen-maintained environment after the medical device has been sterilized through a compartment of the packaging and then sealed.

Herein, the pouch 1 is configured with five (5) layers to accomplish the above. In an exemplary embodiment, the pouch 1 is generally rectangular. In an example embodiment, the length of the pouch 1 is approximately 13″, and the width of the pouch 1 is approximately 10″.

All dimensions recited are examples only and greater or lesser are contemplated. More particularly, a base layer 2 has a base periphery 3, a base back end 4 and opposing base outer edges 5. Base layer 2 is a non-breathable, preferably semi-rigid platform comprised of foil, for example, on which the medical device would rest. This can be a metal foil or other metal foil/polymer laminate, thus a first layer. In some embodiments, the metal foil is an aluminum foil or aluminum-formed layer.

The second layer is a breathable first web 6 on the base layer 2. First web 6 has a first web back end 7 and a first web front end 8, the breathable first web 6 adapted to permit a sterilization gas to pass therethrough. By “breathable”, this means the first web 6 preferably is comprised of a synthetic material made from fibers which are spun-bonded to have a permeability or porosity to gas or vapor. An example of such breathable material includes a spun-bonded olefin such as Tyvek® and paper. Paper may include any thin, flexible material made from a pulp adapted to be coated on some of its surfaces with an adhesive. Other breathable membranes may be used. The Tyvek® properties include high-density polyethylene (HDPE) fibers and allow for a generally tear-proof barrier. Such barrier prevents microbe ingress while concurrently allowing for the passage of air due to its slight porosity. For instance, this first web 6 is adapted to permit pass therethrough of a sterilization gas while filtering out larger microbes. In the exemplary embodiment the first web 6 is approximately 2″ in width.

A third layer is a non-breathable middle film 9 which is substantially co-planar to first web 6 and has a first edge 10, a second edge 11 and opposing film side edges 12. The first edge 10 of the middle film 9 is adhered and sealed to the first web 6. “Non-breathable” means the middle film 9 is made of polymeric material which prevents the ingress of any air and acts as a barrier to any contact by a neighboring component. Here, heat sealable polymers, such as nylons and polyolefins, may form the middle film 9, much like a laminate.

Fourth, a breathable second web 13 is attached to the middle film 9 substantially co-planar therewith as well, having a web rear edge 14, a web front edge 15 and opposing web side edges 16. Second web 13 has similar properties as the first web 6, being preferably made of Tyvek®, hence its breathability. The second edge 11 of the middle film 9 is adhered and sealed to the web rear edge 14 of second web 13 as shown. The first edge 10 of the middle film 9 and the first web front end 8 of the first web 6 are also sealed off.

Fifth, a non-breathable top layer 17 overlaps and is removably stacked over the base and thereby over all of the aforementioned layers, for example 10.125″ in length and thus slightly set back from the length of the base layer 2. Top layer 17 has a top periphery 18, a film rear edge 19, a film front edge 20 and opposing top layer side edges 21. Top layer 17 may be made of materials similar to middle film 9 and/or of base layer 2. In some embodiments, the top layer 17 is a metal foil layer, such as, for example an aluminum foil or an aluminum-formed layer. In some embodiments, the top layer 17 is a non-breathable polymer, such as, for example, a transparent non-breathable polymeric material so that the contents of the pouch 1 may be visible.

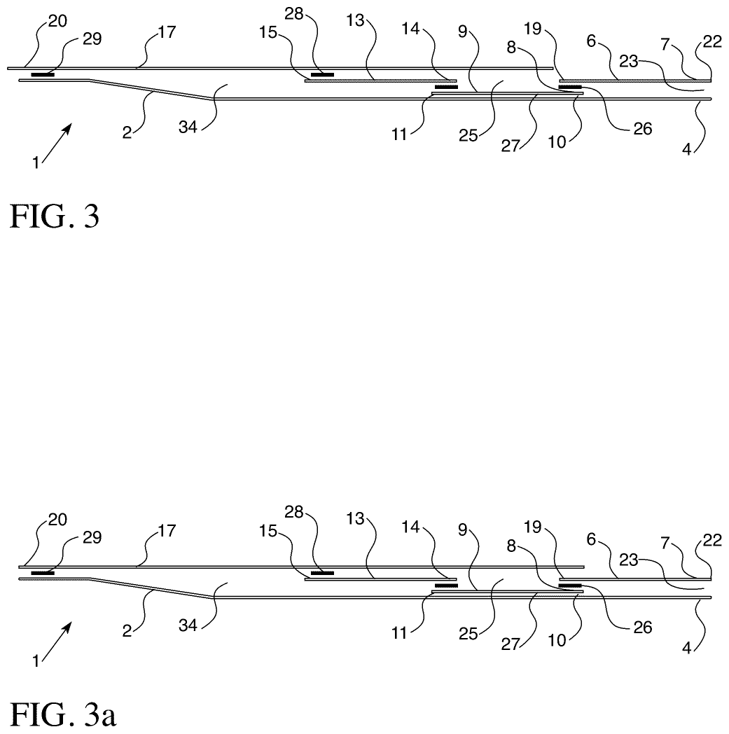

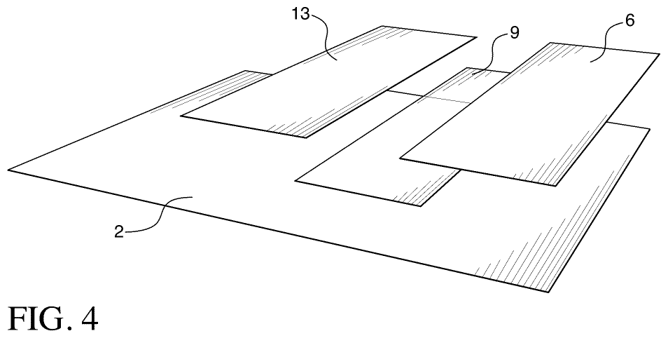

All of the five layers of pouch 1 now stacked, a back edge 22 is defined between the first web back end 7 and the base back end 4, the back edge 22 forming a mouth 23 through which a device, such as a medical device, may be inserted. The back edge 22 is scalable by a sterilization seal 24 (see FIG. 7). The second edge 11 of middle film 9 underlays the top layer 17. In this manner, a desiccant pocket 25 is formed between the middle film 9 and the top layer 17 into which a desiccant can be disposed. The desiccant pocket 25 is sealable by a first closing seal 26 and the second edge 11 (of middle film 9) and the first web front end 8 are sealed off. The second edge 11 of the middle film 9 is adhered to the web rear edge 14 of the second web 13 but forms a breathable interface 27 (see FIG. 3) between the desiccant pocket 25 and the second web 13.

“Pocket” as used herein mean one or more of the compartments that are formed from building out the layers, e.g. desiccant pocket 25, main pocket 34, etc., hence “one or more” pockets. In continuing to put the five webs, or layers, together, sealing occurs to adhere, connect or attach each component or compartment to thereby define the one or more pockets. For instance, a third seal 28 adheres the web front edge 15 to the top layer 17, thereby forming a path through second web 13 for ambient air dried by the desiccant to enter main pocket 34.

A seal around the periphery of the pouch 1 joins and seals most components. This peripheral seal comprises a primary seal 29 and a secondary seal 30. More particularly, the primary seal 29 joins and seals the base layer 2 and the top layer 17 along the top periphery 18 and the base periphery 3 along a first portion of the seal width. This primary seal 29 overlaps an entirety of the base outer edges 5 as shown to seal the top layer side edges 21. The secondary seal 30 of that same peripheral seal adheres the middle film 9 and the second web 13 to the base layer 2 at the base outer edges 5 between the base layer and top layer 17. Note the secondary seal 30 overlaps the primary seal 29 an entire length of the primary seal 29 between the web front edge 15 and the first web front end 8 at the top periphery 18 and the base periphery 3. The secondary seal 30 also captures all of the layers at an overlap location 31 of the first closing seal 26 (FIG. 2). The overlap location 31 is therefore the portion of “double-sealing” where the secondary seal 30 and primary seal 29 are entirely co-extensive, i.e. extend and overlap equally for both width and length throughout this segment. In contrast, the secondary seal 30 partially overlaps the primary seal 29 at a partial overlap portion 32 distal to the first closing seal 26 across a portion of the peripheral seal width to thereby form a setback 33 from the base outer edges 5. Partial overlap portion 36 therefore is the portion of “double-sealing” where the secondary seal 30 and primary seal 29 are only partially co-extensive, i.e. overlapping as to length only, thus the lesser width exposes a partial width of the primary seal 29 and is therefore set back at this setback 33 location. Note the partial overlap portion 32 is considered distal to either the first closing seal 26 or the overlap location 31. Additionally, the setback 33 is proximate to the first edge 10 of middle film 9 at first closing seal 26. “Proximate” means co-linear or nearly co-linear with first edge 10, i.e. slightly offset from first edge 10 at first closing seal 26 as shown in FIGS. 2 and 2a for example. The setback 33 allows the first web 6, the middle film 9 and the second web 13 to be captured between the base layer 2 and top layer 17 in a single sealing step, while also providing a region of direct sealing between base layer 2 to top layer 17 as part of that same primary seal 29. As a result, a sterile main pocket 34 is efficiently formed between the base layer 2 and the top layer 17 adapted to receive and contain a medical device, the medical device also now retained within a stable environment formed by the combination of the five layers.

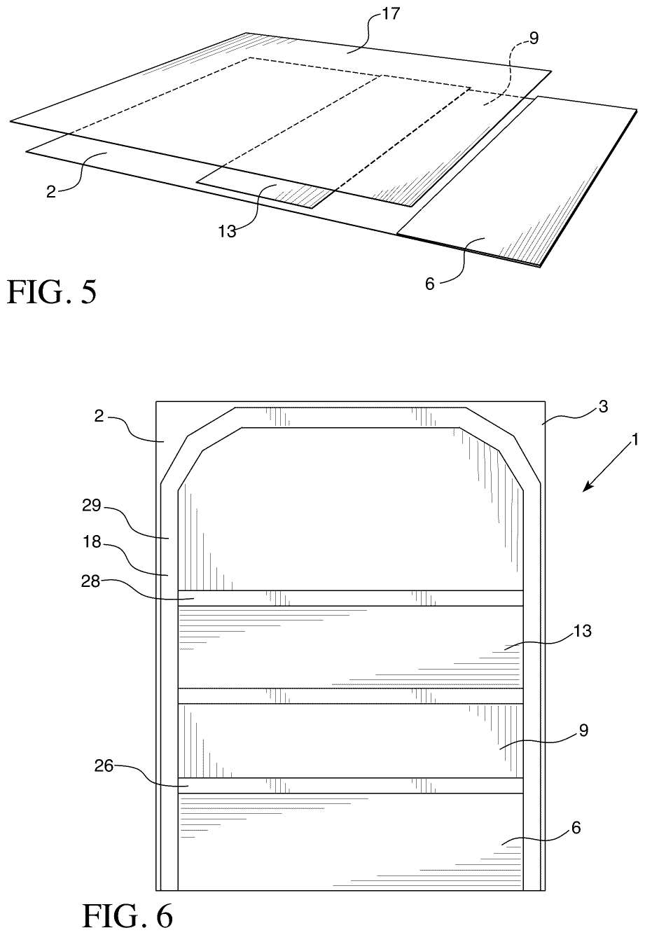

In a process of manufacturing the pouch 1, the base layer 2, the first web 6, the middle film 9, the second web 13, and the top layer 17 are all fed simultaneously from one or more machines, such as, for example, a primary machine and an auxiliary machine, to form a film assembly as shown in FIG. 5. When the base layer 2, the first web 6, the middle film 9, the second web 13, and the top layer 17 are arranged in their appropriate relative locations, the primary seal 29 is formed by applying sufficient heat and sufficient pressure for a sufficient amount of time at the location of the primary seal 29, such as, for example, by an aluminum tool, to cause adjacent layers to adhere, either based on an adhesive layer on one or both of the adjacent layers or based on the material of one or both of the layers itself. In a similar manner, the secondary seal 30 and the third seal 28 are formed. The primary seal 29, the secondary seal 30, and the third seal 28 may be formed separately or simultaneously in a single sealing step.

The desiccant (not shown), or any type of oxygen scavenger, can be inserted into desiccant pocket 25 (between webs 6, 13). This allows the desiccant to perform the function of drying air within the pouch 1 while also not directly contacting the underlying medical device.

Although described herein as being well-suited to contain a medical device, the pouch 1 may alternatively or additionally contain any other appropriate item that would benefit by the features and advantages provided by the main pocket 34 or any other pocket of the pouch 1.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A pouch, comprising:
a base layer having a base periphery, a base back end and opposing base outer edges;
a breathable first web on said base layer having a first web back end and a first web front end and adapted to permit a sterilization gas to pass therethrough;
a non-breathable middle film adhered to said first web having a first edge, a second edge and opposing film side edges;
a breathable second web attached to said middle film having a web rear edge, a web front edge and opposing web side edges; and,
a non-breathable top layer over said base layer, thereby said pouch includes five layers, said top layer having a top periphery, a film rear edge, a film front edge and opposing top layer side edges, thereby forming a back edge defined between the first web back end and the base back end, wherein a main pocket is formed between said base layer and said top layer.

2. The pouch of claim 1, further comprising:
said second edge of said middle film adhered to said first web, and said second edge underlaying said top layer; said first edge of said middle film adhered to said second web, thereby forming a desiccant pocket between said middle film and said top layer into which a desiccant can be disposed.

3. The pouch of claim 2, wherein said desiccant pocket is sealed by a first closing seal such that said film rear edge, said second edge and said first web front edge are sealed off.

4. The pouch of claim 2, further comprising said first edge of said middle film adhered to said web rear edge of said second web thereby forming a breathable interface between said desiccant pocket and said second web.

5. The pouch of claim 2, further comprising a third seal adhering said web front edge to said top layer thereby forming a path for ambient air dried by said desiccant.

6. The pouch of claim 1, further comprising a primary seal adhering said base layer and said top layer along said top periphery and said base periphery, said primary seal overlapping an entirety of said base outer edges to seal said top layer side edges.

7. The pouch of claim 6, further comprising a secondary seal adhering said middle film and said second web to said base layer at said base outer edges.

8. The pouch of claim 7, further comprising said secondary seal being coextensive with said primary seal at an overlap location, said secondary seal extending a partial width of said primary seal at a partial overlap portion distal to said overlap location to thereby form a setback from said base outer edges, wherein said setback allows said first web, said middle film and said second web to be formed in a single sealing step.

9. The pouch of claim 8, wherein said setback is proximate to said first edge of said middle film.

10. A pouch, comprising:
a base layer having a base periphery, a base back end and opposing base outer edges;
a non-breathable top layer having a top periphery, a film rear edge, a film front edge and opposing top layer side edges;
a primary seal adhering said base layer and said top layer along said top periphery and said base periphery, said primary seal overlapping an entirety of said base outer edges to seal said top layer side edges, thereby defining one or more pockets between said base layer and said top layer, said one or more pockets including a desiccant pocket defined between a middle film and said top layer into which a desiccant can be disposed; said desiccant pocket sealed by a first closing seal transverse to said base outer edges;
a secondary seal adhering said one or more pockets to said base layer at said base outer edges, said secondary seal being coextensive with said primary seal at an overlap location of said first closing seal, said secondary seal extending a partial width of said primary seal at a partial overlap portion distal to said first closing seal to thereby form a setback from said base outer edges, wherein said setback allows said one or more pockets to be formed in a single sealing step.

11. The pouch of claim 10, wherein said one or more pockets includes a breathable first web on said base layer having a first web back end and a first web front end and adapted to permit a sterilization gas to pass therethrough.

12. The pouch of claim 11, wherein said one or more pockets includes a non-breathable middle film adhered to said first web having a first edge, a second edge and opposing film side edges.

13. The pouch of claim 12, wherein said one or more pockets includes a breathable second web attached to said middle film having a web rear edge, a web front edge and opposing web side edges.

14. The pouch of claim 13, wherein said first edge of said middle film is adhered to said web rear edge of said second web but forming a breathable interface between said desiccant pocket and said second web.

15. The pouch of claim 13, further comprising a third seal adhering said web front edge to said top layer thereby forming a path for ambient air dried by said desiccant.

16. A pouch, comprising:
a base layer;
a breathable first web on said base layer having a first web back end and a first web front end and adapted to permit a sterilization gas to pass therethrough;
a non-breathable middle film adhered to said first web;
a breathable second web attached to said middle film having a web front edge;
a non-breathable top layer over said base layer;
a primary seal adhering said base layer and said top layer along a top periphery and a base periphery; and,
a secondary seal adhering said middle film and said second web to said base layer, said secondary seal overlapping said primary seal an entire length of said primary seal between said web front edge and said first web front end at said top periphery and said base periphery.

17. The pouch of claim 16, further comprising a desiccant pocket defined between a middle film and said top layer into which a desiccant can be disposed; said desiccant pocket being sealable by a first closing seal transverse to said base outer edges.

18. The pouch of claim 17, further comprising a third seal adhering said web front edge to said top layer thereby forming a path for ambient air dried by said desiccant.

19. The pouch of claim 16, wherein said secondary seal is coextensive with said primary seal at an overlap location of a first closing seal transverse to said base outer edges, said secondary seal extending a partial width of said primary seal at a partial overlap portion distal to said location of said first closing seal to thereby form a setback from said base outer edges.

20. The pouch of claim 19, wherein said setback is proximate to a first edge of said middle film.

21. A process of manufacturing a pouch comprising a base layer; a breathable first web on said base layer having a first web back end and a first web front end and adapted to permit a sterilization gas to pass therethrough; a non-breathable middle film adhered to said first web; a breathable second web attached to said middle film having a web front edge; a non-breathable top layer over said base layer; a primary seal adhering said base layer and said top layer along a top periphery and a base periphery; and a secondary seal adhering said middle film and said second web to said base layer, the process comprising:
simultaneously feeding said base layer, said breathable first web, said non-breathable middle film, said breathable second web, and said non-breathable top layer to form a film assembly; and
forming said primary seal and said secondary seal in a single sealing step by applying pressure and heat to said film assembly, said secondary seal overlapping said primary seal an entire length of said primary seal between said web front edge and said first web front end at said top periphery and said base periphery, thereby forming said pouch.
